(19) United States
(12) Patent Application Publication
(10) Pub. No.: US 2023/0152319 A1
(43) Pub. Date: May 18, 2023

(54) DEVICE AND METHOD FOR RAPID DETECTION OF VIRUSES

(71) Applicants: TECHNION RESEARCH AND DEVELOPMENT FOUNDATION LTD., Haifa (IL); NANOSE MEDICAL LTD, Isfiya (IL)

(72) Inventors: Hossam HAICK, Haifa (IL); Orit MAROM ALBECK, Herzliya (IL); Osnat SELLA TAVOR, Kfar Kisch (IL)

(21) Appl. No.: 17/996,680

(22) PCT Filed: Apr. 21, 2021
(86) PCT No.: PCT/IL2021/050451

§ 371 Date: Oct. 20, 2022

Related U.S. Application Data
(60) Provisional application No. 62/704,097, filed on Apr. 21, 2020.

(51) Int. Cl. G01N 33/569; G01N 33/543; A61B 5/08

(52) U.S. Cl. CPC G01N 33/56983; G01N 33/54346; A61B 5/082; G01N 2333/165; A61B 2562/0285

(57) ABSTRACT

The invention proposes an approach utilizing novel and artificially intelligent hybrid sensor arrays with multiplexed detection capabilities for disease-specific biomarkers from the exhaled breath of a subject. The technology provides a rapid and highly accurate diagnosis in various COVID-19 infection and transmission scenarios.

### TECHNOLOGICAL FIELD

The technology generally concerns a device and method for rapid detection of viruses, more specifically corona viruses, by detection of biological markers in a subject's exhaled air.

### BACKGROUND

Coronavirus-19 disease (COVID-19) is an infectious disease caused by a newly discovered coronavirus called SAES-CoV-2. The virus originated probably in bats and was transmitted to humans through yet unknown intermediary animals in Wuhan, Hubei province, China in December 2019. The coronavirus disease is transmitted mostly by inhalation or contact SARS-CoV-2 infected surfaces or contact with infected droplets from saliva or discharge from the nose and the incubation period ranges from 2 to 14 days. The symptoms are usually fever, cough, sore throat, breathlessness, fatigue, malaise among others. The disease is mild in most people; in some (usually the elderly and those with comorbidities like cardiovascular disease, diabetes, chronic respiratory disease, and cancer) it may progress to pneumonia, acute respiratory distress syndrome (ARDS) and multi organ dysfunction. COVID-19 can be severe, and some cases have caused death although many people are asymptomatic.

COVID-19 is presently diagnosed and confirmed with a molecular test. While the molecular tests are accurate and considered to be the gold standard for SARS-CoV-2 testing, they require obtaining swab sample from the subject and a time-consuming laboratory procedure. Shipping of samples and overload of the laboratory facilities lead to a delay of multiple days until test results are available, thus increasing the burden on the healthcare system. Furthermore, the sensitivity of the tests is high only for those who already have symptoms due to a high load of the virus; however, it is already known that the disease can be spread by asymptomatic carriers that only show mild or even no symptoms at all.

Volatile organic compounds (VOCs) constitute endogenous products of metabolic processes. VOCs are transported from different organs via blood to the lungs and subsequently excreted from the lungs by diffusing across the pulmonary alveolar membrane and exhaled via the breath.

### GENERAL DESCRIPTION

The inventors of the technology disclosed herein have devised a non-invasive, rapid and easy-to-use tool for diagnosing or monitoring infection by COVID-19 at an early stage of infection, even before symptoms of COVID-19 are manifested.

The invention proposes an approach that utilizes novel and artificially intelligent hybrid sensor arrays with multiplexed detection capabilities for disease-specific biomarkers from the exhaled breath of a subject. The technology provides a rapid and highly accurate diagnosis in various COVID-19 infection and transmission scenarios. The breath-based approach relies on a handheld analyzer that collects exhaled breath sample, e.g., by blowing into the device for 2-3 seconds from a distance of 2-3 cm (or by blowing directly to a disposable collecting tube such as an inlet soft tube), and, thereafter, analyzes the COVID-19 status within 2-3 minutes.

This breath analyzer may be designated for Point-of-Care (PoC) centers (clinics, hospitals), central facilities (airports, public places), or as active-case searching in the community, for early detection of the disease in asymptomatic contagious individuals. Due to its relatively low cost and ease of use, it can be used also for continuous monitoring purposes, e.g., individuals at homes that are found in an isolation period and protection of medical teams. Continuous real-time monitoring of COVID-19 is instrumental in stratifying patient risk and elaborating the IC concept to manage COVID-19-care and to mitigate COVID-19 impact. The rationale behind this approach relies on robust findings showing that infectious agents and/or their microenvironment emit volatile organic compounds (VOCs). A part of these VOCs appear in the exhaled breath. The emergence of the VOCs in these body fluids occurs in very early stages of the infection. Therefore, their detection can serve for early detection of the COVID-19.

The invention incorporates secure transmission components to enable ethical and privacy-ensured diagnosis and monitoring by physicians, national health systems and worldwide health organizations. By creating a sample database, predictive models can be established for predicting disease development among the high-risk groups and hospitalization periods and prognosis for positive patients. This thus enable not only adequate patient diagnosis, treatment, and follow-up, but also a continual screening of at-risk populations and real-time monitoring of epidemics, providing population-wide and location-based data for statistical analysis and data mining, and thereby facilitating the in-depth epidemiological study.

Thus, according to a first aspect, the present invention provides a method of identifying presence of a viral infection in a subject (wherein the subject is optionally asymptomatic), the method comprising:

a) determining a volatile organic compound (VOC) profile in a breath sample from the subject; and

b) comparing the VOC profile to a VOC profile of a control, as defined herein, and/or to a VOC profile obtained from the subject at an earlier time point(s), as defined herein;

wherein in case the VOC profile from the breath sample is different from that obtained from the control or for the subject at an earlier time, a determination can be made, as explained herein, that one or more of (1) presence of a viral infection, and (2) absence of a viral infection is true.

In a method of the invention, determining the VOC profile involves exposing the breath sample to a sensor surface comprising a plurality of nanoparticles, e.g., metallic nanoparticles such as gold nanoparticles or others, that are surface associated with one or more ligand molecules. The sensor surface may comprise one or more sensing regions, each of the regions being associated with same or different population of nanoparticles, such that a signal may be independently derived from each of the sensing areas, and be indicative of an interaction (or lack thereof) between VOCs present the sample and the nanoparticles on the sensing regions.

Each of the sensing regions present on the sensor surface comprises a plurality of nanoparticles of a particular population, wherein each population differs from another in at least one of particle size, particle morphology (e.g., core/shell particles, non-core/shell particles, spherical, cubic, tetrahedral, triangular, dumbbell, elongated, multiparticles or fused particles, etc), particle composition (e.g., doping, metallic particles, non-metallic particles, conductive particles, novel metal particles, hybrid materials, etc), surface decoration (e.g., presence of material islands, association with ligand groups, etc) and others.

In some embodiments, each sensing region comprises a different selection of nanoparticles. In some embodiments, each sensing region comprises a mixed population (an inhomogeneous population) of nanoparticles, while in other embodiments-each sensing region comprises a uniform population (a homogenous population) of nanoparticles.

In a plurality of such sensing regions, which may be set on a single substrate or set on separate spaced apart substrates or on an arrayed substrate, one or more thereof may comprise a plurality of particle populations, namely an inhomogeneous population of particles, wherein some of the nanoparticles differ in structure, others in composition and still others in surface decoration. For example, a sensing region may comprise two populations of nanoparticles, one population comprising particles of one metal and another population comprises particles of a different metal. In a similar way, all particles may be of one metal but differ from each other in their surface decoration (e.g., presence of ligands or selection of ligands).

In some embodiments, the nanoparticles are core/shell particles, non-core/shell particles, spherical, cubic, tetrahedral, triangular, dumbbell, elongated or fused particles. In some embodiments, the particles are spherical in shape.

In some embodiments, the nanoparticles are metallic nanoparticles; wherein the metal is optionally selected amongst any metal of the Periodic Table of the Elements. In some embodiments, the metals are of any of Groups IIIB, IVB, VB, VIB, VIIB, VIIIB, IB and JIB of block d of the Periodic Table. In some embodiments, the metal is selected from Sc, Ti, V, Cr, Mn, Fe, Ni, Cu, Y, Zr, Nb, Tc, Ru, Mo, Rh, W, Au, Pt, Pd, Ag, Au, Al, Mn, Co, Cd, Hf, Ta, Re, Os, Jr and Hg.

In some embodiments, the metal is gold, silver, nickel, cobalt, copper, palladium, platinum or aluminum. In some embodiments, the nanoparticles are gold nanoparticles.

The metallic nanoparticles may or may not be doped or further comprise an amount of another metallic or non-metallic material. The metallic nanoparticles may be bare, namely uncoated, or coated with a plurality of surface associated ligand molecules. Such ligand molecules may have surface anchoring groups which may vary based on, e.g., the composition of the nanoparticles. For example, where the nanoparticles are gold nanoparticles, the surface anchoring groups may be a thiol, a disulfide, an amine and others as known in the art.

In accordance with methods and devices of the invention, nanoparticles utilized are typically metallic particles, as selected, having a coating of at least one ligand molecules on their circumference. The coating may be homogenous, namely with the same ligand or inhomogeneous, namely with a mixture of different ligand molecules.

The inventors of the technology disclosed herein have demonstrated that a sensing sensitivity may be dramatically increased when using a sensor surface with one or more sensing regions associated with nanoparticles having ligand molecules associated thereto. In some embodiments, the ligand molecules may be selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethpxythiolphenol, 4-tert-methylbenzenethiol and 1-heptanethiol.

Thus, in some aspects of the invention a method of the invention aims at identifying the presence of a viral infection in a subject, the method comprising:

a) exposing a breath sample obtained from the subject to a sensor surface comprising a plurality of nanoparticles surface-associated with a ligand selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol,

b) determining a volatile organic compound (VOC) profile indicative of presence one of more VOCs in the breath sample from the subject; and

b) comparing the VOC profile of the subject to a VOC profile of a control and/or to a VOC profile obtained from the subject at an earlier time point(s);

to thereby determine one or more of (1) presence of a viral infection, (2) absence of a viral infection, (3) reoccurrence of the viral infection, (4) the type of viral infection, or (5) viral load or stage of the viral infection.

In some embodiments, the ligand molecules are selected from dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol.

In some embodiments, the ligand molecules are selected from 4-tert-methylbenzenethiol, tert-dodecanethiol and hexanethiol.

In some embodiments, the ligand molecules are 4-tert-methylbenzenethiol and/or tert-dodecanethiol and/or hexanethiol.

In some embodiments, the ligand molecule is tert-dodecanethiol.

In some embodiments, the ligand molecules are selected from butanethiol, dibutyl disulfide, hexanethiol, 1-heptanethiol, tert-dodecanethiol, 2-ethylhexanethiol, 4-tert methylbenzenethiol, 3-ethpxythiolphenol and 4-chlorobenzenemethanethiol.

In some embodiments, the ligand molecules are selected from tert-dodecanethiol, hexanethiol, 2-ethylhexanethiol, 4-tert methylbenzenethiol, 3-ethpxythiolphenol, 4-chlorobenzenemethanethiol, dodecanethiol, and decanethiol.

In some embodiments, the ligand molecules are selected from 2-nitro-4-trifluoro-methylbenzenethiol and benzylmercaptan.

In some embodiments, a sensor surface comprises a plurality of (or one or more) sensing regions, each of the sensing regions is in the form of (or comprises or consists) a plurality of gold nanoparticles, each of the nanoparticles being surface-associated with ligand molecules selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol.

In some embodiments, the gold nanoparticles are surface associated with ligand molecules selected from dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol.

In some embodiments, the gold nanoparticles are surface associated with ligand molecules selected from 4-tert-methylbenzenethiol, tert-dodecanethiol and hexanethiol.

In some embodiments, the gold nanoparticles are surface associated with 4-tert-methylbenzenethiol and/or tert-dodecanethiol and/or hexanethiol.

In some embodiments, the gold nanoparticles are surface associated with tert-dodecanethiol.

In some embodiments, the gold nanoparticles are surface associated with ligand molecules selected from butanethiol, dibutyl disulfide, hexanethiol, 1-heptanethiol, tert-dodecanethiol, 2-ethylhexanethiol, 4-tert methylbenzenethiol, 3-ethpxythiolphenol and 4-chlorobenzenemethanethiol.

In some embodiments, the gold nanoparticles are surface associated with ligand molecules selected from tert-dodecanethiol, hexanethiol, 2-ethylhexanethiol, 4-tert methylbenzenethiol, 3-ethpxythiolphenol, 4-chlorobenzenemethanethiol, dodecanethiol, and decanethiol.

In some embodiments, the gold nanoparticles are surface associated with ligand molecules are selected from 2-nitro-4-trifluoro-methylbenzenethiol and benzylmercaptan.

In some embodiments, the sensor surface comprises at least three sensing regions, each of the sensing regions comprising a different population of nanoparticles, each population is different from another in the surface associated ligand molecules. The ligands are selected as above. In some embodiments, each of the three sensing regions is characterized by gold nanoparticles which are associated to:

in one of the regions to 4-tert-methylbenzenethiol;

in the second region to tert-dodecanethiol; and

in the third region to hexanethiol.

Where the sensor surface comprises a single sensing region, gold nanoparticles at that region are surface associated with tert-dodecanethiol.

In some embodiments, the sensor surface comprises at least 8, 9, 10, 11, 12, 13 or more sensing regions, wherein nanoparticles, gold nanoparticles, at each of these regions are associated to different ligands selected from those mentioned herein.

In some embodiments, the sensor surface comprises at least 8 sensing regions, wherein gold nanoparticles at each of these at least 8 regions are associated to different ligands selected from dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol.

In some embodiments, the sensor surface comprises at least 13 sensing regions, wherein gold nanoparticles at each of these at least 13 regions are associated to different ligands selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethpxythiolphenol, 4-tert-methylbenzenethiol and 1-heptanethiol.

Thus, methods of the invention may utilize a sensor surface with one or a plurality of sensing regions to determine presence or absence of a volatile organic compound (VOC) in a breath sample, and thereby determine a VOC profile indicative of the viral infection, as disclosed herein; wherein each of the sensing region(s) is in the form or comprises or consists a plurality of ligand associated gold nanoparticles selected as disclosed herein. Such methods and devices permit rapid and accurate (low false negatives/false positives) assessment of onset of viral infections even where symptoms are yet to appear. An accuracy of at least 84% has been determined by utilizing methods and devices according to the invention.

Methods of the invention may be carried out on a subject suspected of having been infected with a virus. Typically, the subject is asymptomatic, namely showing no symptoms associated with the viral infection. In some embodiments, the method is carried out on a subject showing symptoms indicative of viral infection, wherein the method is used to determine whether the viral infection is by SARS-CoV-2, causing COVID-19.

A breath sample is obtained from a subject by employing any non-invasive means known in the art. In some embodiments, a breath sample is obtained by direct exhalation of breath into a device configured and operable for such a purpose. In some embodiments, a sample is taken from a subject by suction or other means. In cases where cooperation from the subject cannot be obtained, e.g., elderly subjects, disabled subjects, children, hospitalized subjects and others, breath samples may be obtained without needing the subject's cooperation by suction or by any other invasive or non-invasive means. Non-limiting methods for collecting such exhaled breath may involve the use of apparatuses approved by the American Thoracic Society/European Respiratory Society (ATS/ERS), see for example Silkoff et al., Am. J. Respir. Crit. Care Med., 2005, 171, 912.

A viral infection generally refers to a condition whereby cells are invaded by a pathogenic virus, causing a cascade of medical events that can lead to death. The virus is any pathogenic virus such as those selected from coronaviridae/corona-virus, orthomyxoviridae, paramyxoviridae, Coxsackie family of viruses and adenoviridae family. In some embodiments, the virus is a corona-virus, such as a COVID-19 causing pathogen. In some embodiments, the COVID-19 causing pathogen is SARS-CoV-2, encompassing SARS-CoV-2 having mutations that may be found in the entire genome of SARS-CoV-2 strains, e.g., in the 5′ UTR, ORFlab polyprotein, intergenic region, envelope protein, matrix protein and nucleocapsid protein.

In some embodiments, methods of the invention are directed at the diagnosis of COVID-19.

As noted hereinabove, methods of the invention are based on the ability to detect certain VOCs indicative of manifestation of a viral infection in a subject.

Within the context of the present invention, volatile organic compounds (VOC) are compounds, typically but not necessarily organic compounds, that are associated with the metabolism, presence and/or growth of at least one viral pathogen involved in the pathogenic manifestation of a viral disease. VOCs that are generated in the body, e.g., through the metabolism of cells or pathogens within the body, are released into the circulatory system and thereafter excreted through the exhaled breath. The VOCs may comprise a plurality of compounds, some of which gaseous, others may be liquids (at a physiological temperature), which are released into the exhaled breath and carried by the breath gases or small droplets of water, and thus can be detected and quantified. Without wishing to be bound by experimental limitations and current knowledge relating to VOCs that are associated with a particular viral disease in a subject, presence of the following non-limiting VOCs may be indicative of a viral infection: ethanal, acetone, octanal, butanone, 2-butanone, methanol, isoprene, heptanal, propanol, propanal, ethyl butanoate, butyraldehyde, isopropanol, methylpent-2-enal, 2,4-octadiene 1-chloroheptane, nonanal and others.

The “VOC profile” refers to the breath signature of the disease, namely to a collection of properties relating to the VOC content of the exhaled breath obtained from a subject. These collective properties are unique and informative, thus may be regarded as a fingerprint or a signature indicating onset, evolution or progression of a certain viral infection over another. The VOC profile differentiating one virus over the other can also provide an insight as to the state of the disease or the progression thereof, can identify the onset of the disease at an early stage before symptoms develop and can assist in determining success of a therapeutic treatment (prophylaxis or treatment of existing symptoms). The properties may be one or more of:
presence or absence of one or more VOCs indicative of the presence of the virus,
the concentration (or amount) of the one or more VOCs,
the presence or absence of other VOCs in combination,
the ratio amounts between the various VOCs, and
a change in the presence or amount of one or more VOCs over time.

As used herein, a “control” which the VOC profile is compared to is any component of a VOC profile obtained from subjects not having a viral infection, namely subjects who have been tested and found not to have been infected by a virus, or known to be free of the virus, or who have a low viral load, as well as from subjects who are symptomatic due to the viral infection. These may be used to define a “healthy group” namely a group of subjects who do not have the infection, and a “sick group”, namely a group of subjects who are symptomatic.

Control samples obtained for the purpose of determining the presence or absence of a viral infection are typically taken from a plurality (one or more) of subjects which have been identified as healthy or as sick. The number of subjects may be at least 10, 20, 30, 40, 50, 60, 70, 80, 90, 100, 150, 200, 250 to thousands of subjects.

Where aiming at determining progression of the disease, one or more VOC profiles may be obtained for a group of subjects suffering from a disease, wherein each profile is obtained at a different time point along the way to recovery.

Where the subject is a human, the control is a human, and where the method if used on non-human mammals, the control group should include species from the same group.

In some embodiments, the VOC profile is detected by an E-nose or according to the method described in US 2012/0326092, herein incorporated by reference. As noted therein, a change in the VOC profile, e.g., as compared to a control, may be determined by utilizing an algorithm such as, but not limited to, artificial neural networks, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART) and statistical methods including, but not limited to, principal component analysis (PCA), partial least squares (PLS), multiple linear regression (MLR), principal component regression (PCR), discriminant function analysis (DFA) including linear discriminant analysis (LDA) or cluster analysis including nearest neighbor.

Using such algorithms or others known in the art, a VOC profile may be regarded as significantly different and thus be indicative of any one of presence or absence of one or more VOCs indicative of the URTI, concentration (or amount) of the one or more VOCs, presence or absence of other VOCs in combination, ratio amounts between the various VOCs and a change in the presence or amount of one or more VOCs over time. The term “significantly different” as used herein generally refers to a quantitative difference in the concentration or level of each VOC from the set or combinations of VOCs as compared to the levels of VOCs in control samples obtained from, e.g., individuals not having a URTI. A statistically significant difference can be determined by any test known to the person skilled in the art. Common tests for statistical significance include, among others, t-test, ANOVA1 Kruskal-Wallis, Wilcoxon, Mann-Whitney and odds ratio. Individual samples (of unknown status) can be compared with data from the reference group (negative control). An increase or decrease in the level as compared to a control or reference value or mean control level or reference value, or a change, difference or deviation from a control or reference value, can be considered to exist if the level differs from the control level or reference value, by about 5 percent or more, by about 10 percent or more, by about 20 percent or more, or by about 50 percent or more compared to the control level or reference value. Statistical significance may alternatively be calculated as P<0.05. Methods of determining statistical significance are known and are readily used by a person of skill in the art. In a further alternative, increased levels, decreased levels, deviation, and changes can be determined by recourse to assay reference limits or reference intervals. These can be calculated from intuitive assessment or non-parametric methods. Overall, these methods calculate the 0.025, and 0.975 fractiles as 0.025*(n+1) and 0.975*(n+1). Such methods are well known in the art. The presence of a VOC marker which is absent in a control, is also contemplated as an increased level, deviation or change. The absence of a VOC marker which is present in a control, for example, is also contemplated as a decreased level, deviation or change.

Various other algorithms are known in the art, which are disclosed, for example, in U.S. Pat. Nos. 6,411,905, 6,606,566, 6,609,068, 6,620,109, 6,767,732, 6,820,012 and 6,839,636, each of which being incorporated herein by reference.

In accordance with the present invention, the VOC profile indicative of a particular virus comprises VOCs that are present in breath samples of subjects having symptoms associated with an infection by the particular virus, in levels which are at least one standard deviation [SD] larger or smaller than their mean level in breath samples of a negative control population. In some embodiments, the levels of VOCs in breath samples are at least 2 standard deviations [SD] or 3[SD] larger or smaller than their mean level in breath/saliva/nasal secretion samples of a negative control population. Accordingly, individual samples (of unknown status) are considered to belong to a sick population when the level of VOCs is at least 1[SD], 2[SD] or 3[SD] larger or smaller than the mean level of VOCs in breath samples of a negative control population.

In some embodiments, the level of the one or more VOC in the sample is significantly increased as compared to the level of the VOC in a control. In some other embodiments, the level of the one or more VOC in the sample is significantly decreased as compared to the level of the VOC in a control. In some embodiments, the levels of the one or more VOC in the sample collected from the patient having viral infection form a pattern which is significantly different from the pattern of the VOCs in the control. In some embodiments, the levels of the one or more VOC in the sample collected from the patient having a viral infection form a pattern which is significantly different from a predetermined pattern of occurrence of VOCs in breath samples taken from subjects who are not suffering from the disease.

In accordance with the present invention, the difference in the VOC profile between a sample collected from a patient suspected of having a viral infection and a control or a sample collected from a healthy patient can be analyzed using various pattern recognition analyzers commonly used in the art and as described, for example, in US 2012/0326092 which is herein incorporated by reference.

Also provided herein is a VOC from breath samples for use in diagnosis, prognosis and/or monitoring of a viral infection, monitoring disease progression and treatment efficacy. The diagnosis, prognosis and/or monitoring of infection comprises the diagnosis of a subject who is at risk of developing the infection, a subject who is suspected of having an infection, or a subject who was diagnosed with the infection using commonly available diagnostic tests.

The invention further provides a device for carrying out methods according to the invention. The device of the invention may be any device for measuring/detecting components of exhaled breath of a subject which comprises a collection chamber for collecting, holding or communicating a volume of an exhaled breath to a sensor surface, as defined, that can produce a unique (e.g. electronic) fingerprint to enable the determination of a VOC profile from breath samples as described herein. Some non-limiting examples of sensor surfaces that can be used in accordance with the present invention includes functionalized surface regions (wherein such surfaces are functionalized with metal nanoparticles, functional molecules, hollow fibers and others), sensors having a functionalized nanowire or a nanotube, a polymer-coated surface acoustic wave (SAW) sensors, sensor employing a semiconductor gas sensor technology, aptamer biosensors, amplifying fluorescent polymer (AFP) sensors and others.

In accordance with the present invention, the sensor may commercially be referred to as an “artificial nose” or as an “electronic nose” which can non-invasively measure at least one VOC in the exhaled breath and/or monitor the concentration of at least one VOC in the exhaled breath of a subject as described herein. Thus, the herein described sensors enable qualitative and/or quantitative analysis of volatile compounds (e.g. gases, vapors, or odors) hence facilitates the device to carry out a method of the invention.

In some embodiments, the device comprises one or more (an array) of chemically sensitive sensing regions and a processing unit comprising a learning and pattern recognition analyzer configured for receiving sensor output signals and comparing the signals to a stored data, by utilizing a pattern recognition algorithm.

In some embodiments, the device may be a device disclosed in International Publication No. WO 2009/144725, herein incorporated by reference. In some embodiments, the device utilizes a sensor as disclosed in US 2011/0269632, herein incorporated by reference.

In some embodiments, devices of the invention utilize sensor surfaces in the form of (or which comprise or consist) a plurality of metal nanoparticles, as disclosed herein.

Thus, the invention further provides a device, optionally a handheld device, for diagnosing, screening or monitoring a viral infection in an asymptomatic subject, the device comprising:
a sensor surface comprising one or more sensing regions, each of the sensing regions comprising ligand-coated (or ligand-associated) nanoparticles, optionally metallic nanoparticles, configured and operable for interacting with one or more VOCs present in the subject's exhaled breath; and
a processing unit comprising a learning and pattern recognition analyzer configured for receiving output signals from the one or more of the sensing regions and comparing the signals to a stored data, by utilizing a pattern recognition algorithm.

In some embodiments, each of the one or more sensing regions is a sensor device arranged in an array form on a sensor surface or a substrate, forming together the sensor unit.

In some embodiments, the device further comprises a signal transmitting module and optionally a user interface. In some embodiments, the device comprises a processor and a user interface. In some embodiments, the signal transmitting module is set for delivering signal parameters or signal output to a processor (data processing unit) and a data user interface for delivering the results and insights. The processor and user interface are optionally part of the device.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts a patient enrolment and observational design.

FIG. 2 shows a representative response of a sensor according to the invention to three different breath samples.

FIG. 3 is an example of breath collection with a novel handheld breathalyzer system constructed according to the invention, from a patient in Wuhan, China.

FIGS. 4A-D provide diagnosis of COVID-19 patients based on cumulative breath sample response according to the invention, as explained herein.

### DETAILED DESCRIPTION OF EMBODIMENTS

The outbreak of severe acute respiratory syndrome coronavirus-2 (SARS-CoV-2, aka COVID-19) has emerged very rapidly and has invaded more than 197 countries worldwide. About 97.5% of patients develop symptoms of COVID-19 within 11.5 days of exposure, causing late diagnosis and a high infection rate. The molecular tests used so far to confirm COVID-19 are accurate and considered the gold standards for SARS-CoV-2 testing. Nevertheless, they require a swab sample and a time-consuming laboratory procedure. Shipping of samples and overload of laboratory facilities entail a delay of many days until the test results are available, increasing the burden on the healthcare system. Furthermore, the tests are highly sensitive only for those who already have symptoms because of the high virus load; however, it is already known that the disease can be spread by asymptomatic carriers who show only mild symptoms or even none at all. For example, RT-PCR analysis takes two days with approximately 70% sensitivity and a high false-negative rate, implying many type 2 errors, causing sick people to be misdiagnosed, resulting in further spread of the disease. Epidemiological data based on the sequenced viral RNA show that the spread of COVID-19 has resulted from local community transmission, which means that the source of infection cannot be traced back to a known exposure. Thus, healthcare systems worldwide require tests that are non-invasive, rapid, inexpensive, and easy-to-use for diagnosing or ruling out infection at earlier stages, even before COVID-19 symptoms manifest, to decrease the transmission and mortality rates.

The device and methods of the invention provide a rapid non-invasive approach that could potentially serve as an epidemic control tool. This will allow a quick response to threats and streamlining of the necessary resources. The approach relies on novel and artificially intelligent hybrid sensor arrays with multiplexed detection capabilities for a COVID-19-specific breath-print pattern in exhaled breath. To test the feasibility of the exhaled breath approach, as a pre-screening diagnostic system, a case-control study was conducted at the origin of the COVID-19 outbreak, Wuhan, China, during March 2020.

A sensor utilized according to the invention is characterized by a surface comprising a plurality of (or one or more) sensing regions, each of the sensing regions is in the form of (or comprises or consists) a plurality of nanoparticles, each of the nanoparticles being surface-associated with ligand molecules. Ligand molecules used in accordance with the invention included one or more of the ligands: dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol.

In some cases, the nanoparticles were gold nanoparticles.

For some measurements, the nanoparticles were surface associated with ligand molecules selected from dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol. For other measurements, the gold nanoparticles were surface associated with ligand molecules selected from 4-tert-methylbenzenethiol, tert-dodecanethiol and hexanethiol, or those selected from 4-tert-methylbenzenethiol and/or tert-dodecanethiol and/or hexanethiol.

In some cases, the nanoparticles were surface associated with tert-dodecanethiol.

In other cases, the nanoparticles were surface associated with ligand molecules selected from butanethiol, dibutyl disulfide, hexanethiol, 1-heptanethiol, tert-dodecanethiol, 2-ethylhexanethiol, 4-tert methylbenzenethiol, 3-ethpxythiolphenol and 4-chlorobenzenemethanethiol.

In some other cases, the nanoparticles were surface associated with ligand molecules selected from tert-dodecanethiol, hexanethiol, 2-ethylhexanethiol, 4-tert methylbenzenethiol, 3-ethpxythiolphenol, 4-chlorobenzenemethanethiol, dodecanethiol, and decanethiol, or selected from 2-nitro-4-trifluoro-methylbenzenethiol and benzylmercaptan.

Methods

Study Population #1

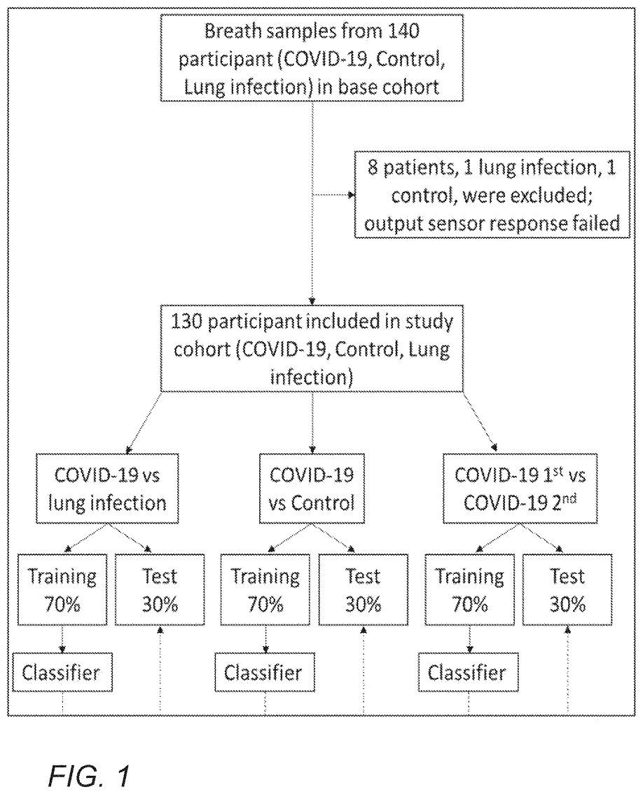

A total of 140 participants were enrolled at multiple centers in Wuhan and Hefei, China, as part of an observational study: 49 COVID-19 patients, 58 healthy controls and 33 non-COVID lung infection controls (FIG. 1). The COVID-19 patients were confirmed by computed tomography (CT), nasal and pharyngeal swab specimens for real-time reverse-transcriptase polymerase-chain-reaction (RT-PCR), and antibody tests. The enrolled COVID-19 patients were sampled at two time points approximately 3-5 days apart in the COVID-19 intensive care ward assigned to the First Affiliated Hospital of USTC entrusted by the State Council of China located in Wuhan Union Hospital, China. The research protocol was approved by the ethics committee of Anhui Provincial Cancer Hospital, West District of The First Affiliated Hospital of USTC. All participants provided written informed consent.

Breath Analyzer

Portable hand-held and notebook computer-connected artificially intelligent hybrid sensor arrays with multiplexed detection capabilities were used in the current project. The principle of breath analysis in these devices is the change in resistance of the sensors when they come into contact with a particular mixture of VOCs. This allows the device to be trained to recognise a particular disease, COVID-19 in our case. The sensor array used in the device contained cross-reactive, chemically diverse chemiresistors based on organically stabilized spherical gold nanoparticles (GNPs) developed by TECHNION (Haifa, Israel). The measurement protocol comprised two steps: baseline reading and reading from the real breath sample. Therefore, two sets of data from each of the eight sensors in the array were obtained from each study subject using the two devices.

Breath Sampling

Breath samples were collected by the study subjects breathing directly into the aperture of the instrument for at least four seconds, keeping the instrument approximately 1-2 cm from the mouth. Built-in sensor technology advised the study subject when the test was complete. If the breath collection was not satisfactory, the subject was asked to repeat the test.

Feature Extraction and Statistical Analysis

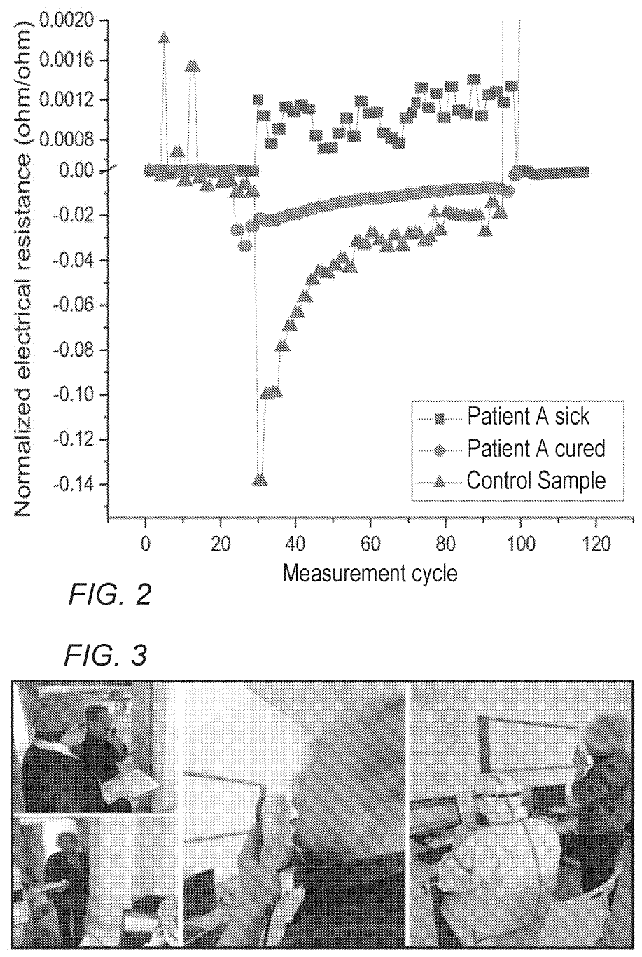

Features were extracted from the output files of the breath samples for all sensors in the array. An example of a sensor response to different samples can be found in FIG. 2. The feature was calculated as the change in electrical resistance between the breath signal and the baseline signal divided by the baseline signal. The tested groups were subjected to binary comparison and the data were divided randomly into training sets (70% samples) and test sets (30% samples). The results of discriminant function analysis (DFA) on the training set were validated using the test set. The base analysis between the COVID-19 and control samples used the quadratic DFA model based on the measurements of three sensors. For the other two sub-comparisons (COVID-19 vs other lung infections; COVID-19 first vs second sampling), the same three sensors as for main model were used by applying two binary linear DFA models. The model performance of the training set was first determined by measuring the area under the curve (AUC) of the receiver operating characteristic (ROC) and was used to calculate the cut-off values on the basis of Youden's index, which classifies the tested groups as giving either a positive or a negative result for the test set classification. Subsequently, other parameters of model performance were analyzed including accuracy, sensitivity, specificity, positive predictive value (PPV), and negative predictive value (NPV). To address the influence of the main confounding factors on breath analysis, age, gender and smoking status were registered and evaluated on the basis of the same model classifier achieved by DFA. The first and second samplings from the same COVID-19 patients (i.e., correlated variables) were compared using paired t-test analysis (by subject) only between patients who had provided samples at the two time points. The matched platform results with a Tukey mean-difference presented the mean difference and 95% confidence interval (CI). Significant differences in the one-way and/or matched pair test were considered at a cut-off p-value of 0.05 between the sub-groups checked, as determined from the results using JMP Pro, version 14.0.0 (SAS Institute Inc., Cary, N.C., USA, 1989-2005). Of the 140 samples, 10 were excluded after collection for technical reasons, either bad sampling or sensor failure during sampling and before statistical analysis.

In FIG. 2, the normalized response of sensor 7 of the breathalyzer system to three different samples: Patient A, COIVD-19 first sample while sick; Patient A, second sample after determined as cured; and healthy control. The X-axis represents cycle measurement; each unit is one cycle of the sensor. The sick sample had a positive change response while the cured and control showed negative charges.

Results

Participants

The selection of participants from three distinct groups—COVID-19 patients, healthy controls, and non-COVID lung infection controls—is described in FIG. 1. The characteristics of the 140 participants are shown in Table 1. Among the patients, more than 60% had no underlying chronic disease while all the rest suffered at least from hypertension; 10% of those suffered from diabetes mellites. Most patients were nonsmokers (73%), with a mean age of 59 years with 57% females. For the control group, 67% were nonsmokers, and the mean age was 52 years with 46% females. For the lung infection control group, 73% were nonsmokers, and the mean age was 63 years with 44% females.

TABLE 1
Demographics, baseline characteristics and laboratory testing of study
population. Patients positive for COVID-19 were identified prior to
breath sampling. Only the COVID-19 group was accessed for
molecular and immunological characteristics. IQR-interquartile range,
CT-computed tomography, RT-PCR-real-time reverse transcriptase
polymerase chain reaction.

| | | | Lung |
|---|---|---|---|
| | COVID-19 | Control | infections |
| | group | group | group |
| Characteristics | (N = 49) | (N = 58) | (N = 33) |
| Median age (IQR)-yr | 58 | 53 (45-59) | 64 (53-73) |
| | (51.5-64.5) | | |
| Female sex-no. (%) | 28 (57) | 26 (45) | 15 (45) |
| Active Smoking-no. (%) | 13 (27) | 19 (33) | 9 (27) |
| Coexisting conditions-no. (%) | | | |
| Hypertension | 18 (37) | 8 (12) | 8 (24) |
| Diabetes | 5 (10) | 2 (3) | 4 (12) |
| Heart disease | 1 (2) | — | 2 (6) |
| Malignancy | — | — | 8 (24) |
| Bacterial\fungal infection | — | — | 6 (18) |
| Other | 1 (2) | — | — |

Means of positive determination COVID-19-no. (%)

| | |
|---|---|
| CT angiogram | 33 (67) |
| RT-PCR, positive | |
| (throat swab)-no. (%) | |
| nCovORFlab | 29 (59) |
| 2019nCOV-N | 28 (57) |

2019nCov antibody tests, median (IQR)-AU/ml

| | |
|---|---|
| 2019nCov IgM | 42.85 |
| | (13.65-69.24) |
| 2019nCov IgG | 164.68 |
| | (133.93-182.55) |

Count of irregularities in immune system component levels-no. (%)

| | |
|---|---|
| CD3 (58.17-84.22%) | |
| Count of high | 7 (14) |
| Count of low | 3 (6) |
| CD4 (25.34-51.37%) | |
| Count of high | 13 (27) |
| Count of low | 1 (2) |
| CD8 (14.23-38.95%) | |
| Count of high | 4 (8) |
| Count of low | 4 (8) |
| CD4/CD8 (0.41-2.72) | |
| Count of high | 12 (25) |
| Count of low | — |

Breath Analysis

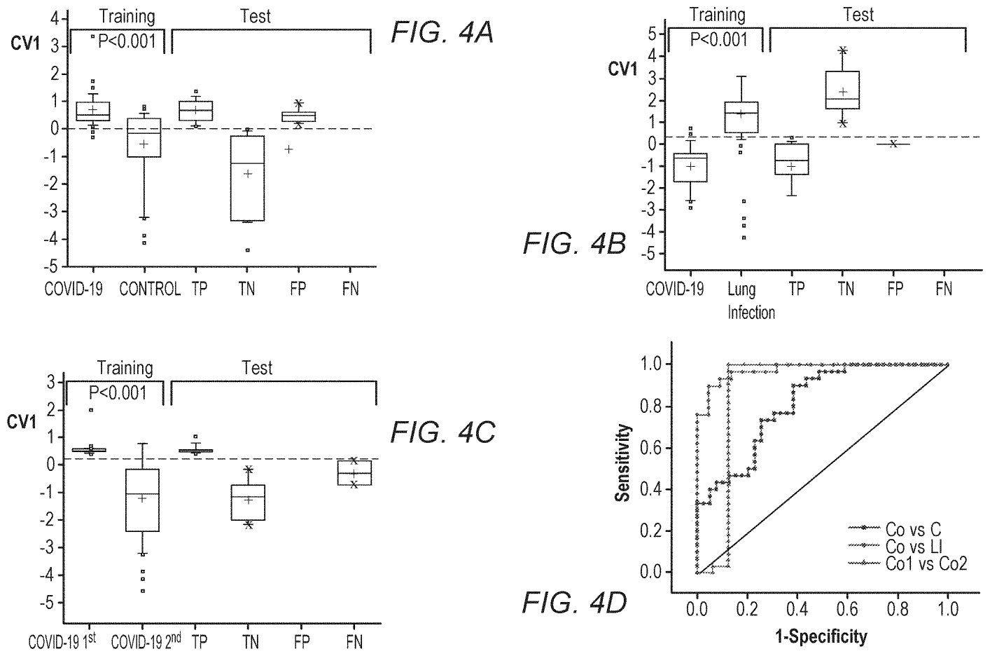

Breath samples were collected and analyzed using a PoC handheld system (FIG. 3). Statistical analysis involved three binary comparisons: COVID-19 vs. control; COVID-19 vs. other lung infections; and COVID-19 1^{st} vs. COVID-19 2^{nd} sample. Seventy percent of the data were used to calculate the DFA models; a ROC analysis was done and the cut-off was determined. The remaining test data were classified on the basis of the cut-off as presented (FIG. 4). Accuracies for the training set varied between 90% and 94% for the three models, and between 76% and 95% for the test set (Table 2). The main comparison between COVID-19 and controls gave 100% sensitivity for both training and test groups with a miss rate of zero, while the false-positive rate was 39%. The paired analysis of the two sample times showed a clear distinction for the measured breath composition score (P<0.001, mean difference (95% CI)=−1.57 (−2.27−−0.87)): in the first sample, all were COVID-19 positive; at the second sample time, all but three were considered cured. Among the three uncured, two were identified by the model as false-negative and one was correctly classified as positive. Confounding effects on the main classification were examined: there were no significant difference in respect of age, gender, smoking status or coexisting conditions. In addition, we tested the plausibility of the main model built to distinguish COVID-19 from controls to classify all subgroups. The model showed significant differences between COVID-19 and control, COVID-19 and cured COVID-19 samples, lung infection and control, and lung infection and cured COVID-19 samples.

FIGS. 4A, B and C show data classification from cumulative sensor responses to breath samples as represented by the canonical variable of the discriminant analysis. Box plots of the first canonical score of the training set (70% of samples) and test set (30% of samples). The horizontal dashed line in the box plots represents the cut-off value of the model. True positive (TP), True negative (TN), False positive (FP), False negative (FN). FIG. 4A: COVID-19 patients (41 participants) and healthy controls (57 participants). FIG. 4B: COVID-19 patients (41 participants) and other lung infection\condition controls (32 participants). FIG. 4C: COVID-19 patients at first sampling (41 participants) and second sampling (21 participants); and COVID-19 patients at second sampling uncured (three participants), and controls who had never smoked and were not identified as having chronic bronchitis by either questionnaire (58 participants). P values are for the comparisons of the training set for each of two binary classifications. The horizontal line in the boxes represents the median, the cross represents the mean, and the bottom and top of the boxes represent the 25th and 75th percentiles, respectively. I bars represent the upper 90 percentile and the lower 10 percentile, and the square-dots outliers. All P values were adjusted for multiple comparisons using the Tukey-Kramer method. For FIG. 4C, the P value is also adjusted for paired analysis. FIG. 4D shows receiver-operating-characteristic (ROC) curves for the cumulative breath-sensor response in patients with defined COVID-19 (Co) infection compared with controls (black curve; area under the curve [AUC], 0.81 [0.70 to 0.89]), in COVID-19 infection compared with other lung infection\conditions (red curve; AUC, 0.97 [0.92 to 0.99]), and in COVID-19 infection first sample compared to COVID-19 infection second sample (AUC, 0.87 [0.67 to 1.00]).

TABLE 2
Breath test outcomes for the study population.

| | Training set | | | Testing set^{‡} | | |
|---|---|---|---|---|---|---|
| | Covid | Covid vs. | Covid1^{st} | Covid | Covid vs. | Covid 1^{st} |
| | vs. | Lung | vs. | vs. | Lung | vs. |
| Statistics | Control^{†} | Infection^{§} | Covid 2^{nd§} | Control | Infection | Covid 2^{nd} |
| Accuracy (%) | 94 | 90 | 90 | 76 | 95 | 88 |
| Sensitivity (%) | 100 | 90 | 100 | 100 | 100 | 83 |
| Specificity (%) | 90 | 91 | 69 | 61 | 90 | 100 |
| PPV (%) | 88 | 93 | 86 | 61 | 92 | 100 |
| NPV (%) | 100 | 87 | 100 | 100 | 100 | 71 |
| TP (cases) | 30 | 26 | 32 | 11 | 12 | 10 |
| TN (cases) | 35 | 20 | 11 | 11 | 9 | 5 |
| FP (cases) | 4 | 2 | 5 | 7 | 1 | 0 |
| FN (cases) | 0 | 3 | 0 | 0 | 0 | 2 |

^{†}Classification based on QDA,
^{§}Classification based on LDA,
^{‡}Classification based on the ROC cut-off.

### DISCUSSION

The results show excellent sensitivities for the three binary comparisons with a minimal false-negative rate, while specificities were above average. The assumption that COVID-19 patients can be differentiated not only from controls but also from other lung conditions/infections was tested. Indeed, the results showed 95% accuracy in classifying the latter conditions. A follow-up of the same patients at a second-time point, when most were already cured, showed 90% accuracy, though two of the three uncured patients were wrongly determined as cured. This could be attributed to the prolonged healing time; it can take a few weeks to reach a definitely cured state. The results are comparable to current published data from COVID-19 studies, which suggest 82%-98% accuracy for abnormal CT findings and 51%-70% sensitivity of the RT-PCR test. The breath test could be affected by such confounding factors as gender, age, smoking status, and coexisting conditions, so it was important to test their influence on results; but no significant influence was found, similar to previous reports on this technology.

It is expected that real-time methods such as the exhaled breath approach reported here will significantly reduce unnecessary exposure to contagious persons and support the fight against the COVID-19 pandemic. Moreover, it will reduce the number of unnecessary confirmatory tests and lower the burden on hospitals, while providing individuals with a screening solution that can be performed at home or in PoC facilities. During hospitalization or home isolation, a combination of a sensing patch and a breath analyzer will serve to monitor treatment success and disease regression.

Study Population #2

The technology is based on a sensor array that measures Volatile Organic Compounds (VOCs) emitted in the exhaled breath, which are biomarkers for a variety of diseases. Artificial Intelligence analyzes the signal pattern to reveal unique “VOC print” for each disease. This test does not require any user special skills and therefore can be accessible at all point of care, and even for home tests.

The aim of the study was defined as: Collecting and evaluating data of potential volatile biomarkers in the exhaled air of subjects with and without Covid-19 by the novel sensors of the invention. COVID-19 positive and negative subjects were enrolled. Classification to the 2 study arms was based on a PCR test result. Three medical centers participate in the study: Shamir Health Corporation (“Assaf Harofeh”) in Israel; Northwell Health, Inc. in the United States (“Northwell”); Zayed Military Hospital Abu Dhabi (“Zayed Hospital”).

The study was performed with the sensors installed in 2 devices: 1. The first-generation device with single use units that include the sensors. 2. a device with multi use sensors. The collected data from the devices were analyzed independently by two distinct methods.

The first dataset was collected with the first-generation device with singe use units that include the sensors of the invention. The dataset included subjects tested with the device at two sites: 35 samples from Northwell N.Y., and 31 samples from Shamir medical center IL. Each test file consisted of responses from duplicated sensor array, and therefore each test file was split into two sample files, based on the sensor sets. Some of the sensors failed to respond, and therefore datasets that included failed sensors were discarded. The total number of sample files that were analyzed after the error-prone samples were discarded is: Northwell—35 sample files (representing 24 tested subjects—17 positives, 7 negatives) and Shamir medical center—31 sample files (representing 21 tested subjects—14 positives, 7 negatives). The data was analyzed by Brainchip with a Spiking Neural Network, the adjacent confusion matrix shows the results on the test set. The test set included 31 samples-21 positives and 10 negatives from 21 tested subjects. Zero out of 21 positive samples were identified correctly which represents 100% sensitivity and 4 out of 10 negative samples were identified correctly which represents 40% specificity. The overall accuracy was 80.65% The second study was performed with the multiuse Nallose sensors installed in Sniffphone device. The dataset included 165 samples taken from 141 subjects tested with Sniffphone device at Zayed Military Hospital—65 samples from 65 COVID-19 positive subjects and 100 samples from 76 COVID-19 negative subjects (Several negative subjects were sampled two or three times). A Linear discriminative analysis was performed. The adjacent confusion matrix shows the results on the test set that that was completely blind to the training and validation of the model. The test set included 37 samples—8 positive and 29 negative samples from 27 tested subjects. Seven out of eight positive samples were identified correctly which represents 87.5% sensitivity, and 25 out of the 29 negative samples were identified correctly which represents 86.2% specificity. The overall accuracy was therefore 86.5%.

The same data set was analyzed also by the SNN methodology. To make the SNN most efficient, 34 samples were discarded due to noise or improper vector dimensionality. Thus, the dataset included 131 samples taken from 126 subjects tested with Sniffphone device at Zayed Military Hospital-62 samples from 62 COVID-19 positive subjects and 69 samples from 64 COVID-19 negative subjects (Several negative subjects were sampled two or three times). The adjacent confusion matrix shows the results on the test set that that was completely blind to the training and validation of the model. The test set included 53 samples—20 positive and 33 negative samples from 53 tested subjects. Nineteen out of 20 positive samples were identified correctly which represents 95% sensitivity and 29 out of 33 negative samples were identified correctly which represents 87.87% specificity. The overall accuracy was therefore 90.5%.

Two different analysis methods were applied on the dataset and both showed excellent results for the differentiation between COVID positive and COVID negative. While the multiuse sensors achieved a much better specificity (˜87%) compared to the single use sensors (40%), this is more likely a result of the vast difference between the datasets: the dataset of the multiuse sensors included 165 samples from 141 subjects while the dataset of the single-use sensors included 66 samples from 45 subjects. During the Clinical study with COVID19 patients the company further improved the 4 components of the device: the mechanical design including the breath collection mechanism, the electronics, the sensors and the classifying algorithm.

1-48. (canceled)

49. A method of identifying the presence of a viral infection in a subject, the method comprising:
a) exposing a breath sample obtained from the subject to a sensor surface comprising a plurality of nanoparticles surface-associated with a ligand selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol,
b) determining a volatile organic compound (VOC) profile indicative of presence one of more VOCs in the breath sample from the subject; and
b) comparing the VOC profile of the subject to a VOC profile of a control and/or to a VOC profile obtained from the subject at an earlier time point(s); to thereby determine one or more of (1) presence of a viral infection, (2) absence of a viral infection, (3) reoccurrence of the viral infection, (4) the type of viral infection, or (5) viral load or stage of the viral infection.

50. The method according to claim 49, wherein the sensor surface comprises one or more sensing regions, each of the sensing regions being associated with same or different independently measurable population of nanoparticles.

51. The method according to claim 50, wherein each of the sensing regions comprises a plurality of nanoparticle populations, wherein each of the plurality of population differs from another in at least one of nanoparticle size, nanoparticle morphology, nanoparticle composition, and surface decoration.

52. The method according to claim 49, wherein the nanoparticles are metallic nanoparticles.

53. The method according to claim 52, wherein the metallic nanoparticles comprise or consist a metal selected from gold, silver, nickel, cobalt, copper, palladium, platinum and aluminum or alloy or metal combination thereof.

54. The method according to claim 53, wherein the nanoparticles are gold nanoparticles.

55. The method according to claim 49, wherein the ligand molecules comprise one or more of dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol.

56. The method according to claim 49, wherein the ligand molecules comprise one or more of 4-tert-methylbenzenethiol, tert-dodecanethiol and hexanethiol.

57. The method according to claim 49, wherein the ligand molecules comprise 4-tert-methylbenzenethiol and/or tert-dodecanethiol and/or hexanethiol.

58. The method according to claim 49, wherein the ligand molecules comprise tert-dodecanethiol.

59. The method according to claim 49, wherein the sensor surface comprises a plurality or one or more sensing regions, each of the sensing regions is in the form of a plurality of gold nanoparticles, each of the nanoparticles being surface-associated with ligand molecules selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol.

60. The method according to claim 59, wherein the gold nanoparticles are surface associated with ligand molecules selected from dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol.

61. The method according to claim 49, wherein the sensor surface comprises at least three sensing regions, each of the sensing regions comprising a different population of nanoparticles, each population differing from another in the surface associated ligand molecules.

62. The method according to claim 61, wherein the sensor surface comprises at least 8 sensing regions, wherein gold nanoparticles at each of the at least 8 regions is associated to different ligands selected from dodecanethiol, 2-ethylhexanethiol, 4-tert-methylbenzenethiol, decanethiol, 4-chlorobenzenemethanethiol, 3-ethoxytiophenol, tert-dodecanethiol and hexanethiol.

63. The method according to claim 61, wherein the sensor surface comprises at least 13 sensing regions, wherein gold nanoparticles at each of these at least 13 regions are associated to different ligands selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethpxythiolphenol, 4-tert-methylbenzenethiol and 1-heptanethiol.

64. The method according to claim 49, wherein the breath sample is obtained from a subject by direct exhalation of breath into a device configured and operable for carrying out the method, or into to a disposable collecting tube optionally in the form of a soft tube, or by suction.

65. A device, optionally in a form of a handheld device, for diagnosing, screening or monitoring a viral infection in an asymptomatic subject, the device comprising:
a sensor surface comprising one or more sensing regions, each of the sensing regions comprising ligand-associated nanoparticles, configured and operable for interacting with one or more VOCs present in the subject's exhaled breath; and
a processing unit comprising a learning and pattern recognition analyzer configured for receiving output signals from the one or more of the sensing regions and comparing the signals to a stored data, by utilizing a pattern recognition algorithm;
wherein the ligands associated to the nanoparticles are selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol.

66. The device according to claim 65, wherein the sensor surface comprises a plurality or one or more sensing regions, each of the sensing regions is in the form of a plurality of gold nanoparticles, each of the nanoparticles being surface-associated with ligand molecules selected from dodecanethiol, hexanethiol, decanethiol, tert-dodecanethiol, butanethiol, 2-ethylhexanethiol, dibutyl disulfide, 2-nitro-4-trifluoromethylbenzenethiol, benzylmercaptane, 4-chlorobenzenemethanethiol, 3-ethoxythiolphenol, 4-tert-methylbenzenethiol and 1-heptanethiol.

67. The device according to claim 65, wherein the breath sample is obtained from a subject by direct exhalation of breath into a device configured and operable for carrying out the method, or into to a disposable collecting tube optionally in the form of a soft tube, or by suction.
